(19) United States
(12) Patent Application Publication
(10) Pub. No.: US 2009/0182197 A1
(43) Pub. Date: Jul. 16, 2009

(54) TOOLS FOR USE IN SMALL INTESTINE

(75) Inventors: Benad Goldwasser, Tel Aviv (IL); Oz Cabiri, Macabim (IL); Yossi Gross, Moshav Mazor (IL); Shlomo Lewkowicz, Tivon (IL)

Correspondence Address: DARBY & DARBY P.C., P.O. BOX 770, Church Street Station, New York, NY, 10008-0770, US

(73) Assignee: G.I. VIEW LTD., Ramat Gan (IL)

(21) Appl. No.: 11/997,666

(22) PCT Filed: Aug. 1, 2006
(86) PCT No.: PCT/IL06/00889

§ 371 Date: Aug. 29, 2008

Related U.S. Application Data
(60) Provisional application No. 60/704,654, filed on Aug. 1, 2005.

(51) Int. Cl. A61B 1/04

(52) U.S. Cl. 600/115

(57) ABSTRACT

Apparatus (10) is provided for use with a biologically-compatible-fluid pressure source. The apparatus includes an inflatable guide member (30), configured to be mounted in a vicinity of a small intestinal site, and to form a pressure seal upon inflation. An elongate carrier (24) is configured to be slidably advanced through the guide member into a small intestine (20) of the subject. An imaging capsule (26) is coupled to a distal portion of the carrier. The imaging capsule includes an imaging element (42) and a piston head (40). The piston head forms a pressure seal with a wall of the small intestine and is advanced distally through the small intestine in response to pressure from the fluid pressure source.

### CROSS-REFERENCES TO RELATED APPLICATIONS

The present application claims the benefit of U.S. Provisional Patent Application 60/704,654 to Goldwasser et al., entitled, “Tools for use in small intestine,” filed Aug. 1, 2005, which is assigned to the assignee of the present patent application and is incorporated herein by reference.

### FIELD OF THE INVENTION

The present invention relates generally to a pressure-propelled system, suitable for imaging body lumens, such as the gastrointestinal (GI) tract.

### BACKGROUND OF THE INVENTION

Many imaging devices are known for producing medical images of body lumens, such as the gastrointestinal (GI) tract. For example, endoscopy is widely used for observing, photographing tissue, and taking specimens from lesions and the like.

US Patent Application Publication 2005/0154355 to Gross et al., which is assigned to the assignee of the present application and is incorporated herein by reference, describes apparatus for use with a fluid pressure source. The apparatus includes an elongate carrier, adapted to be inserted through a proximal opening of a body lumen, and a distal piston head coupled to a distal portion of the carrier. The piston head is adapted to be in direct contact with a wall of the lumen when the carrier is inserted into the lumen, and to be advanced distally through the body lumen in response to pressure from the fluid pressure source.

The following references, which are incorporated herein by reference, may be of interest:

US Patent Application Publication 2004/0102681 to Gross

US Patent Application Publication 2005/0036059 to Goldwasser

US Patent Application Publications 2005/0038318 and 2005/0038319 to Goldwasser

US Patent Application Publication 2005/0038335 to Gross et al.

US Patent Application Publication 2005/0154278 to Cabiri et al.

PCT Publication WO 05/065044 to Cabiri et al.

U.S. Pat. No. 5,984,860 to Shan

U.S. Pat. No. 6,866,626 to Long et al.

U.S. Pat. No. 5,571,114 to Devanaboyina

U.S. Pat. No. 6,682,479 to Takahashi et al.

US Patent Application Publication 2004/0260150 to Bernstein

U.S. Pat. No. 6,709,388 to Mosse et al.

US Patent Application Publication 2005/0095200 to Schwarzberg

US Patent Application Publication 2005/0038317 to Ratnakar

U.S. Pat. No. 6,869,393 to Butler

U.S. Pat. No. 5,941,815 to Chang

U.S. Pat. No. 5,879,325 to Lindstrom et al.

U.S. Pat. No. 5,337,732 to Grundfest et al.

US Patent Application Publication 2003/0168068 to Poole and Young

US Patent Application Publication 2003/0105386 and U.S. Pat. No. 6,485,409 to Voloshin et al.

US Patent Application Publication 2002/0107478 to Wendlandt

U.S. Pat. No. 6,702,735 to Kelly

U.S. Pat. No. 5,259,364 to Bob, et al.

U.S. Pat. No. 4,403,985 to Boretos

U.S. Pat. No. 4,176,662 to Frazer

U.S. Pat. No. 4,148,307 to Utsugi

U.S. Pat. No. 5,906,591 to Dario et al.

U.S. Pat. No. 6,007,482 to Madni et al.

U.S. Pat. No. 5,662,587 to Grundfest et al.

U.S. Pat. No. 4,690,131 to Lyddy, Jr. et al.

U.S. Pat. No. 4,040,413 to Ohshiro

U.S. Pat. No. 6,503,192 to Ouchi

U.S. Pat. No. 6,814,728 to Ouchi

U.S. Pat. No. 6,911,005 to Ouchi et al.

US Patent Application Publication 2003/0083547 to Hamilton et al.

PCT Publication WO 04/069057 to Gobel

US Patent Application Publication 2003/0000526 to Gobel

PCT Publication WO 03/045487 to Gobel

U.S. Pat. No. 4,561,427 to Takada

U.S. Pat. No. 6,071,234 to Takada

U.S. Pat. No. 6,332,865 to Borody et al.

### SUMMARY OF THE INVENTION

In some embodiments of the present invention, an endoscopic imaging system propelled by fluid pressure is provided for examining a small intestine of a subject. The system comprises an inflatable guide member configured to be mounted in a vicinity of an ileocecal valve or a pyloric valve of the subject, so as to form a pressure seal with the valve. The system further comprises an elongate carrier arranged for sliding movement through the inflatable guide member, and an imaging capsule coupled to a distal portion of the carrier. The imaging capsule comprises a piston head and an imaging element. The piston head is configured to form a pressure seal with a wall of the small intestine, and to be advanced distally through the small intestine in response to pressure from a fluid pressure source. The system is typically configured to image an entire length of the small intestine, and, for some applications, to collect a tissue or fluid sample of the small intestine and/or release a drug in the small intestine. The imaging capsule typically remains coupled to the carrier throughout the procedure. Upon conclusion of the procedure, the imaging capsule is typically withdrawn using the carrier, or released from the carrier so that the capsule travels through the gastrointestinal tract and is expelled through the rectum.

In some embodiments in which the inflatable guide member is mounted in a vicinity of the pyloric valve, the imaging capsule and inflatable guide member are configured to be coupled to a distal end of a gastric tube or gastroscope, which is advanced through the stomach to the pyloric valve. For some applications, the gastric tube or gastroscope comprises a distal deflection mechanism for navigating the distal end of the tube or endoscope to the pyloric valve.

In embodiments in which the inflatable guide member is mounted in a vicinity of the ileocecal valve, the imaging capsule and inflatable guide member are configured to be inserted into a rectum of the subject, and advanced through the colon and cecum to the ileocecal valve.

There is therefore provided, in accordance with an embodiment of the invention, apparatus for use with a biologically-compatible-fluid pressure source, including:

an inflatable guide member, configured to be mounted in a vicinity of a valve of a subject selected from the group consisting of: an ileocecal valve and a pyloric valve, and to form a pressure seal upon inflation;

an elongate carrier, configured to be slidably advanced through the guide member into a small intestine of the subject; and

an imaging capsule coupled to a distal portion of the carrier, the imaging capsule including:

an imaging element; and

a piston head, configured to:
form a pressure seal with a wall of the small intestine, and
be advanced distally through the small intestine in response to pressure from the fluid pressure source.

In an embodiment, the selected valve includes the ileocecal valve, and the inflatable guide member is configured to be mounted in a vicinity of the ileocecal valve.

In an embodiment, the selected valve includes the pyloric valve, and the inflatable guide member is configured to be mounted in a vicinity of the pyloric valve.

In an embodiment, the inflatable guide member is configured to be mounted within a duodenal bulb of the subject.

In an embodiment, the inflatable guide member is configured to be mounted outside of the small intestine.

In an embodiment, the inflatable guide member is configured to be mounted within the selected valve and to form the pressure seal, upon inflation, with the selected valve.

There is further provided, in accordance with an embodiment of the invention, apparatus for use with a biologically-compatible-fluid pressure source, including:

an inflatable guide member, configured to be mounted in a vicinity of a small intestinal site, and to form a pressure seal upon inflation;

an elongate carrier, configured to be slidably advanced through the guide member into a small intestine of the subject; and

an imaging capsule coupled to a distal portion of the carrier, the imaging capsule including:

an imaging element; and

a piston head configured to:
form a pressure seal with a wall of the small intestine, and
be advanced distally through the small intestine in response to pressure from the fluid pressure source.

In an embodiment, the capsule includes a sample collection unit, configured to sample fluid or tissue of the small intestine.

In an embodiment, the apparatus includes a sample collection unit coupled to the carrier and not an integral portion of the imaging capsule, wherein the collection unit is configured to sample fluid or tissue of the small intestine.

In an embodiment, the capsule is releasably coupled to the distal portion of the carrier, and the capsule is configured to be released from the carrier while the capsule is in the small intestine.

In an embodiment, the piston head is at least 2 cm from the imaging element.

In an embodiment, the piston head is 3-5 cm from the imaging element.

In an embodiment, the piston head is positioned with respect to the imaging element such that distal motion of the imaging capsule causes cleaning of the imaging element by rubbing of the imaging element against the wall of the small intestine.

In an embodiment, the apparatus includes a vent tube configured to facilitate passage of a fluid from (a) a site distal to the piston head to (b) a site proximal to the piston head.

In an embodiment, the inflatable guide member, is configured to be deflated following being mounted, and to subsequently be inflated at a site distal to where the inflatable guide member had been mounted, to an extent sufficient to form a pressure seal upon inflation.

There is still further provided, in accordance with an embodiment of the invention, a method for use with a biologically-compatible-fluid pressure source, including:

forming a pressure seal at a pressure seal site in a vicinity of a valve of a subject selected from the group consisting of: an ileocecal valve and a pyloric valve;

placing an elongate carrier distal to the pressure seal site, in a small intestine of the subject;

forming a pressure seal between a piston head coupled to the elongate carrier and a wall of the small intestine;

advancing the elongate carrier distally through the small intestine by applying pressure from the fluid pressure source to the piston head; and

imaging the small intestine from a distal portion of the carrier.

In an embodiment, the selected valve includes the ileocecal valve, and forming the pressure seal at the pressure seal site includes forming the pressure seal in the vicinity of the ileocecal valve.

In an embodiment, the selected valve includes the pyloric valve, and forming the pressure seal at the pressure seal site includes forming the pressure seal in the vicinity of the pyloric valve.

In an embodiment, forming the pressure seal at the pressure seal site includes forming the pressure seal within a duodenal bulb of the subject.

In an embodiment, forming the pressure seal at the pressure seal site includes forming the pressure seal outside of the small intestine.

In an embodiment, forming the pressure seal site includes forming the pressure seal within the selected valve.

There is yet further provided, in accordance with an embodiment of the invention, a method for use with a biologically-compatible-fluid pressure source, including:

forming a pressure seal at a pressure seal site within a small intestine of a subject;

placing an elongate carrier distal to the pressure seal site, in the small intestine of the subject;

forming a pressure seal between a piston head coupled to the elongate carrier and a wall of the small intestine;

advancing the elongate carrier distally through the small intestine by applying pressure from the fluid pressure source to the piston head; and

imaging the small intestine from a distal portion of the carrier.

In an embodiment, the method includes sampling fluid or tissue of the small intestine.

In an embodiment, an imaging element for the imaging of the small intestine is coupled to the elongate carrier, and including releasing, in the small intestine, the imaging element from the elongate carrier.

In an embodiment, imaging includes imaging from a site at least 2 cm from the pressure seal between the piston head and the wall of the small intestine.

In an embodiment, imaging includes imaging from a site 3-5 cm from the pressure seal between the piston head and the wall of the small intestine.

In an embodiment, an imaging element for the imaging of the small intestine is coupled to the elongate carrier, and including cleaning the imaging element by rubbing the imaging element against the wall of the small intestine.

In an embodiment, the method includes facilitating passage of a fluid from (a) a site distal to the pressure seal between the piston head and the wall of the small intestine to (b) a site proximal to the pressure seal between the piston head and the wall of the small intestine.

In an embodiment, the pressure seal site defines a first pressure seal site, and the method includes removing the pressure seal at the first pressure seal site following forming the pressure seal at the first pressure seal site, and subsequently forming a pressure seal at a second pressure seal site distal to the first pressure seal site.

The present invention will be more fully understood from the following detailed description of embodiments thereof, taken together with the drawings, in which:

### BRIEF DESCRIPTION OF THE DRAWINGS

FIGS. 1A and 1B are schematic illustrations of an imaging system configured to be inserted into a small intestine of a subject via an intestinal valve, in accordance with respective embodiments of the present invention;

FIGS. 2A and 2B are schematic illustrations of the imaging system of FIGS. 1A and 1B during insertion of the system into the small intestine via a stomach of the subject, in accordance with an embodiment of the present invention;

FIGS. 3A, 3B, and 3C are schematic illustrations of the imaging system of FIGS. 1A and 1B comprising a sample collection unit, in accordance with an embodiment of the present invention; and

FIG. 4 is a schematic illustration of the imaging system of FIGS. 1A and 1B advanced through a colon of the subject to the small intestine, in accordance with an embodiment of the present invention.

### DETAILED DESCRIPTION OF EMBODIMENTS

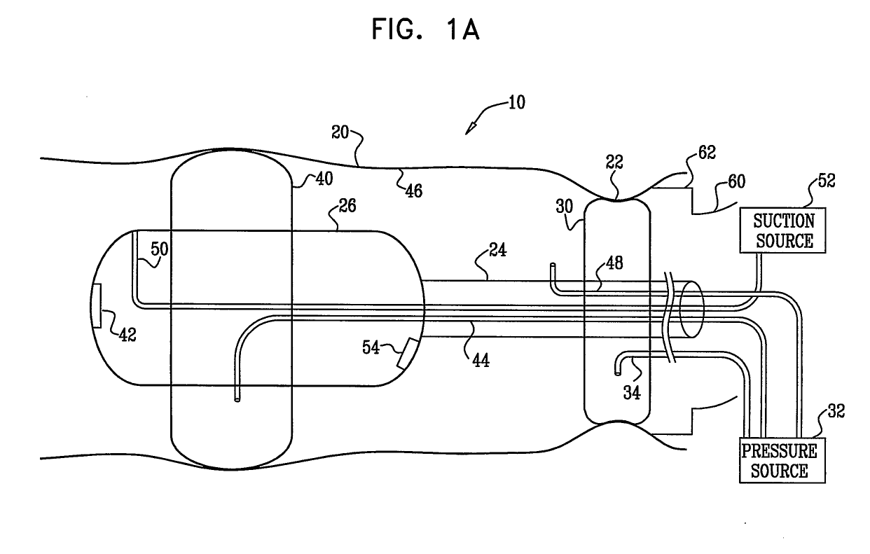

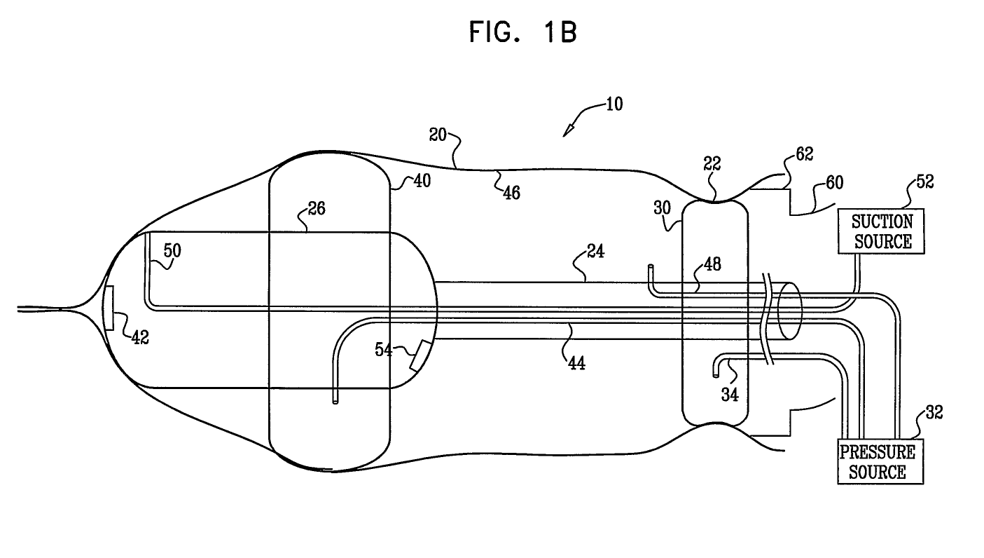

Reference is now made to FIGS. 1A and 1B, which are schematic illustrations of an imaging system 10 configured to be inserted into a small intestine 20 of a subject via an intestinal valve 22, in accordance with respective embodiments of the present invention. System 10 comprises an elongate carrier 24, an imaging capsule 26 coupled to a distal portion of the carrier, and an inflatable guide member 30. Elongate carrier 24 comprises a plurality of tubes and communication wires, as described hereinbelow, and is arranged for sliding movement through guide member 30. Inflatable guide member 30 is configured to be mounted in a vicinity of intestinal valve 22 (either a pyloric valve or an ileocecal valve), so as to form a pressure seal with the valve. For some applications, guide member 30 is placed within valve 22 (as shown). For other applications, guide member 30 is placed within the small intestine, e.g., within the duodenal bulb. For yet other applications, guide member 30 is secured adjacent to valve 22, but outside of small intestine 20.

An interior of guide member 30 is in fluid communication with a pressure source 32 via a guide member fluid supply tube 34. Pressure source 32 provides a pressurized biologically-compatible fluid, such as but not limited to, a source of pressurized air, CO2, or water.

Imaging capsule 26 comprises a piston head 40 and an imaging element 42. Piston head 40 is configured to be inflated in response to pressure from fluid pressure source 32 delivered via a piston fluid supply tube 44. Once inflated, piston head 40 forms a pressure seal with a wall 46 of small intestine 20. Piston head 40 comprises a medically-safe elastomeric material, such as polyurethane or silicone rubber.

For some applications, piston head 40 is disposed near the center of capsule 26 (as shown in FIG. 1A). Alternatively or additionally, the center of piston head 40 is at least 2 cm (e.g., about 3-5 cm) from the most distal portion of capsule 26 and/or at least about 2 cm (e.g., about 3-5 cm) from imaging element 42 (as shown in FIG. 1B). Typically, this positioning is such that distal motion of capsule 26 naturally causes cleaning of imaging element 42 as it slides through the small intestine and rubs against the wall of the small intestine. Alternatively or additionally, other techniques for cleaning imaging element 42 known in the art are utilized.

Piston head 40 is configured to be advanced distally through the small intestine in response to pressure from fluid pressure source 32 delivered, via an advancement fluid supply tube 48, to a volume of small intestine 40 proximal to piston head 40 and distal to inflatable guide member 30. (In this context, in the specification and in the claims, “proximal” means closer to the orifice—mouth or rectum—through which imaging capsule 26 is originally inserted, and “distal” means further from this orifice.)

For some applications, system 10 additionally comprises a vent tube 50 in fluid communication with an area of small intestine 20 distal to piston head 40. The vent tube facilitates passage of fluid (gas and/or liquid) out of the small intestine from the area distal to piston head 40. The vent tube is configured to passively permit the passage of the fluid out of the area, or is coupled to a suction source 52 for actively facilitating the passage of the fluid out of the area.

For some applications, imaging capsule 26 advances through small intestine 20 at a rate of about 10-50 cm per minute. For some applications, imaging capsule 26 is advanced partially through small intestine 20, and guide member 30 is deflated and advanced into the small intestine a portion of the distance to imaging capsule 26. The guide member is subsequently inflated, and the imaging capsule is again advanced. This alternating mode of advancement is repeated until the capsule arrives at the end of the small intestine.

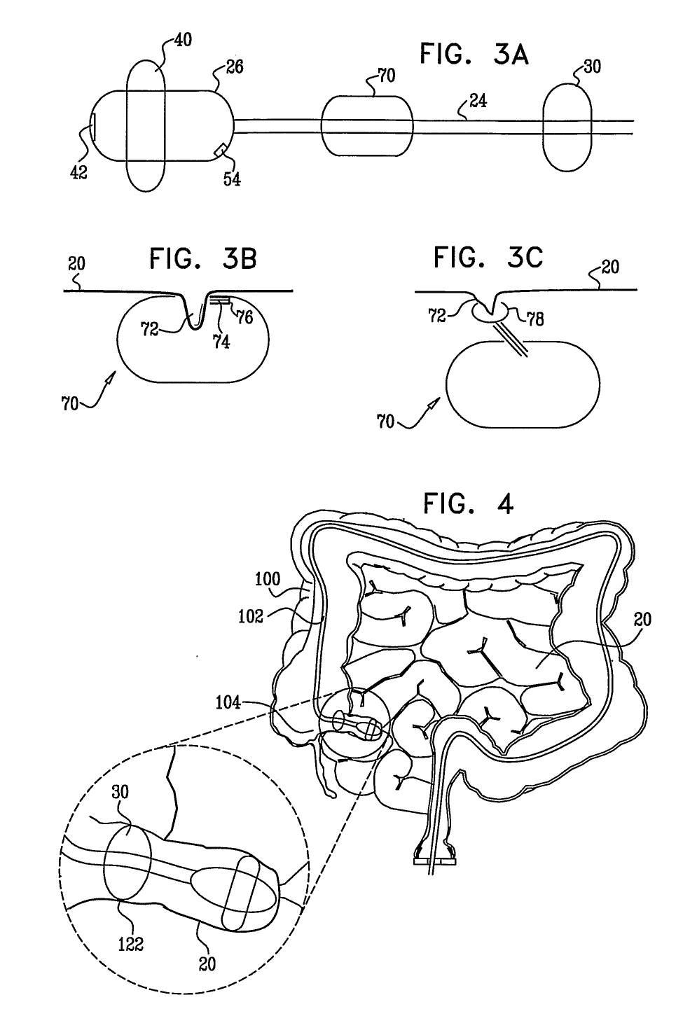

Imaging element 42 comprises a camera (e.g., CCD or CMOS), or an x-ray, ultrasonic, MRI, infrared, and/or microwave imaging device. For some applications, imaging element 42 comprises one or more lens configured to enable forward and omnidirectional viewing, and/or means for illuminating the small intestine. For example, techniques may be used that are described in U.S. Provisional Patent Application 60/571,438, filed May 14, 2004, and/or International Patent Application PCT/IL2005/000500, filed May 11, 2005, both of which are assigned to the assignee of the present application and are incorporated herein by reference. Alternatively or additionally, imaging capsule 26 comprises a rear-viewing imaging element 54, as described in more detail hereinbelow with reference to FIGS. 3A-C.

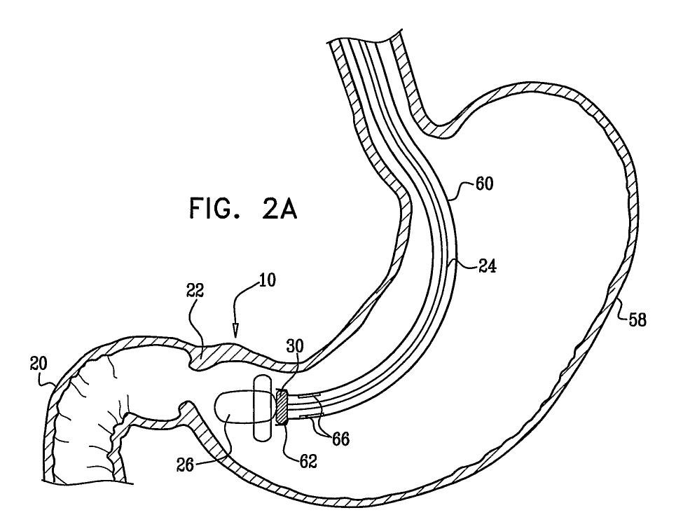

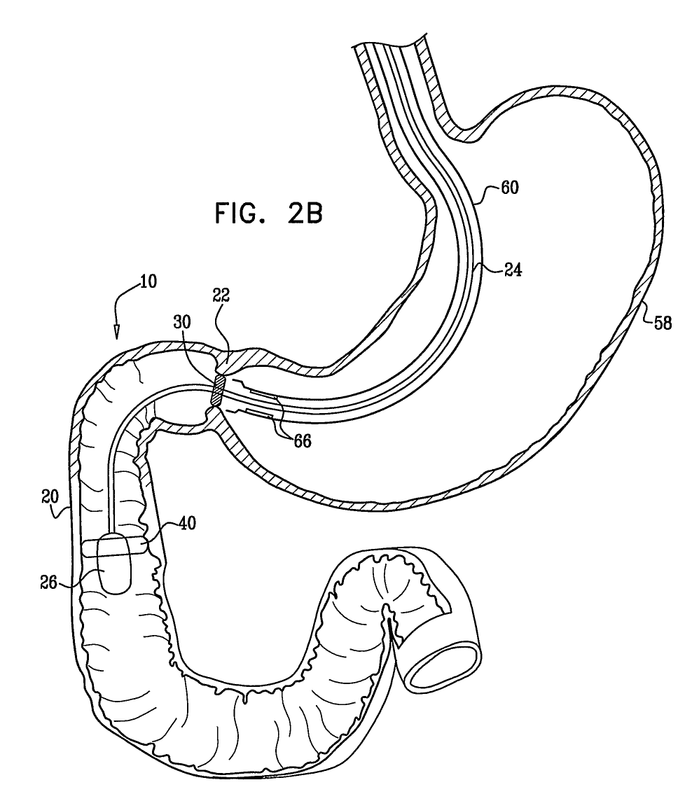

FIGS. 2A and 2B are schematic illustrations of imaging system 10 during insertion of the system into small intestine 20 via a stomach 58 of the subject, in accordance with an embodiment of the present invention. As shown in FIG. 2A, an introducer tube 60 is used to advance imaging capsule 26 and inflatable guide member 30 through stomach 58 of the subject to pyloric valve 22. Introducer tube 60 typically comprises a conventional gastric tube or gastroscope.

For some applications, introducer tube 60 comprises a steering mechanism 66 for deflecting a distal end of the introducer tube, such as is known in the endoscopic and catheter art. For example, steering mechanism 66 may comprise two or more guidewires configured to enable deflection of the distal end of the introducer tube in two or more directions (configuration not shown) . For some applications, images generated by imaging element 42 are used to assist in guiding steering mechanism 66 through stomach 58 to pyloric valve 22.

For some applications, imaging capsule 26 and guide member 30 are configured to be coupled to a distal end of the introducer tube by a coupling element 62. Alternatively, introducer tube 60 is advanced into stomach 58, and imaging capsule 26 and guide member 30 are advanced through the introducer tube, such as by pushing on carrier 24 (configuration not shown).

As shown in FIG. 2B, after inflatable guide member 30 has been positioned in the vicinity of pyloric valve 22, the guide member is inflated, and introducer tube 60 is typically withdrawn from stomach 58. Alternatively, guide member 30 remains coupled to introducer tube 60 even after inflation of the guide member, and the introducer tube remains in stomach 58 throughout the procedure. Imaging capsule 26 is advanced through small intestine 20, as described hereinabove with reference to FIGS. 1A and 1B.

System 10 is typically configured to image an entire length of small intestine 20. Imaging element 42 typically transmits images in real time to an external monitor for viewing by the operator of the system who is performing the procedure. Imaging element 42 typically transmits the images over wires passing through carrier 24 (wires not shown for clarity of illustration). Alternatively, the imaging element wirelessly transmits the images to the external monitor. For some applications, system 10 is configured to collect a tissue or fluid sample of the small intestine, such as described hereinbelow with reference to FIGS. 3A-C, and/or to release a drug in the small intestine. Imaging capsule 26 typically remains coupled to carrier 24 throughout the procedure.

Upon conclusion of the procedure, imaging capsule 26 is typically withdrawn using carrier 24, or released from the carrier so that the capsule travels through the gastrointestinal tract and is excreted through the rectum. Such a release of the capsule may be obtained by applying a current that heats a plastic or other fusing material linking the carrier to the capsule, until the fusing material breaks. Alternatively, the capsule and carrier are held together by a magnetic force, and an electromagnetic pulse is applied to separate the capsule from the carrier. Further alternatively, the capsule and carrier are held together by suction, and the suction is removed in order to separate the capsule from the carrier.

In an embodiment, imaging capsule comprises one or more electrodes configured to stimulate contractile tissue of wall 46 of small intestine 20, so as to propel imaging capsule 26 proximally towards pyloric valve 22. Techniques for such stimulation may be used that are described in the above-mentioned U.S. Pat. No. 6,709,388 to Mosse et al. For some applications, such electrical stimulation techniques are used alternatively or additionally to advance and/or hold the capsule in place in small intestine 20. Capsule 26 may be designed for single use or, alternatively, for multiple uses.

Reference is made to FIGS. 3A, 3B, and 3C. FIG. 3A is a schematic illustration of system 10 comprising a sample collection unit 70, in accordance with an embodiment of the present invention. FIGS. 3B and 3C show details of collection unit 70, in accordance with respective embodiments of the present invention. Sample collection unit 70 is configured to collect a tissue or fluid sample 72 of the small intestine. For example, as shown in FIG. 3B, collection unit 70 may use suction to pull tissue 72 into a collection compartment of unit 70, whereupon the tissue is excised by a cutting instrument 74. The excised tissue is maintained within collection unit 70, and, typically, a portion of unit 70 closes in order to maintain separation of the excised tissue from the surrounding environment. The closure of unit 70 and excision of the tissue may, for some applications, be accomplished by instrument 74, which is typically activated by an actuator 76 under physician control. Alternatively, a suitably-instrumented mechanical arm 78 extends from collection unit 70 and retrieves a sample for biopsy (FIG. 3C).

Upon completion of the procedure, sample collection unit 70 is withdrawn proximally by carrier 24 (even in embodiments in which imaging capsule 26 is released from the carrier). Alternatively, the collection unit is released from carrier 24, allowed to be excreted from the rectum, and collected by the subject for later analysis. For some applications, rear-viewing imaging element 54 is used to observe and facilitate the collection of the sample by sample collection unit 70. Alternatively or additionally, sample collection unit 70 performs analysis (e.g., chemical or optical analysis) of collected samples in situ, such as using techniques known in the art, and, typically, transmits information to a site outside of the patient's body. For example, the information may include raw data or results of analysis, and may be transmitted over wires or wirelessly.

For some applications, functionality described herein with respect to collection unit 70 is implemented in capsule 26.

Reference is made to FIG. 4, which is a schematic illustration of system 10 advanced through a colon 100 of the subject to small intestine 20, in accordance with an embodiment of the present invention. In this embodiment, a colonoscope 102 is used to advance imaging capsule 26 and inflatable guide member 30 through colon 100 and into a cecum 104 of the subject, to ileocecal valve 122. For some applications, colonoscope 102 comprises a conventional endoscope. Alternatively, colonoscope 102 utilizes techniques for advancing through colon 100 described in one or more of the above-mentioned patent application publications to Gross, Gross et al., Goldwasser, and Cabiri et al., and/or in one or more of the patent applications mentioned hereinbelow, mutatis mutandis. For some applications, imaging element 42 is used to observe and facilitate the advancement of the imaging capsule through the colon and/or cecum. After inflatable guide member 30 has been positioned in the vicinity of ileocecal valve 122 (typically within the ileum, e.g., in the terminal ileum), the guide member is inflated. Imaging capsule 26 is advanced through small intestine 20, as described hereinabove with reference to FIGS. 1A and 1B.

Although piston head 40 has been described in embodiments of the present invention as being in direct contact with wall 46 of small intestine 20, the scope of the invention includes establishing contact between the piston head and the wall of the intestine through an intermediary, such as a sheath surrounding the piston head.

The scope of the present invention includes embodiments described in the following applications, all of which are assigned to the assignee of the present application and are incorporated herein by reference. In an embodiment, techniques and apparatus described in one or more of the following applications are combined with techniques and apparatus described herein. In particular, such techniques may be used for advancing imaging capsule 26 through small intestine 20 and/or through colon 100.

US Patent Application Publication 2005/0154355 to Gross et al.

US Patent Application Publication 2004/0102681 to Gross

US Patent Application Publication 2005/0036059 to Goldwasser

US Patent Application Publications 2005/0038318 and 2005/0038319 to Goldwasser

US Patent Application Publication 2005/0038335 to Gross et al.

US Patent Application Publication 2005/0154278 to Cabiri et al.

PCT Publication WO 05/065044 to Cabiri et al.

U.S. patent application Ser. No. 10/967,922 to Cabiri et al., filed Oct. 18, 2004, entitled, “Pressure-propelled system for body lumen”

U.S. patent application Ser. No. 10/523,578 to Gross et al., filed Jan. 28, 2005, entitled, “Self-propelled imaging system”

U.S. Provisional Patent Application 60/571,438 to Dotan et al., filed May 14, 2004, entitled, “Omnidirectional and forward-looking imaging device”

U.S. Provisional Patent Application 60/607,986 to Cabiri et al., filed Sep. 8, 2004, entitled, “Mechanical aspects of pressure-propelled system for body lumen”

U.S. Provisional Patent Application 60/642,245, filed Jan. 6, 2005, entitled, “Gastrointestinal tool over guidewire”

International Patent Application PCT/IL2005/000178 to Goldwasser et al., filed Feb. 10, 2005, entitled, “Gastrointestinal tool over guidewire”

U.S. Provisional Patent Application 60/652,049 to Goldwasser et al., filed Feb. 10, 2005, entitled “Advanced techniques for gastrointestinal tool with guiding element”

U.S. Provisional Patent Application 60/680,074 to Degtiar et al., filed May 11, 2005, entitled, “Disposable endoscope connector”

an international patent application to Dotan et al., filed May 11, 2005, entitled, “Omnidirectional and forward-looking imaging device”

U.S. patent application Ser. No. 10/753,424 to Gross et al., entitled, “Pressure-propelled system for body lumen,” filed Jan. 9, 2004

U.S. Provisional Patent Application 60/704,656 to Goldwasser et al., entitled, “Tools for use in esophagus,” filed Aug. 1, 2005

a PCT patent application to Degtiar et al., entitled, “Disposable endoscope connector,” filed May 11, 2006

a PCT patent application to Cabiri et al., entitled, “Endoscopic measurement techniques,” filed May 11, 2006

a PCT patent application to Goldwasser et al., entitled, “Tools for use in esophagus,” filed on even date herewith.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with a biologically-compatible-fluid pressure source, comprising:
an inflatable guide member, configured to be mounted in a vicinity of a valve of a subject selected from the group consisting of: an ileocecal valve and a pyloric valve, and to form a pressure seal upon inflation;
an elongate carrier, configured to be slidably advanced through the guide member into a small intestine of the subject; and
an imaging capsule coupled to a distal portion of the carrier, the imaging capsule comprising:
an imaging element; and
a piston head, configured to:
form a pressure seal with a wall of the small intestine, and
be advanced distally through the small intestine in response to pressure from the fluid pressure source.

2. The apparatus according to claim 1, wherein the selected valve includes the ileocecal valve, and wherein the inflatable guide member is configured to be mounted in a vicinity of the ileocecal valve.

3. The apparatus according to claim 1, wherein the selected valve includes the pyloric valve, and wherein the inflatable guide member is configured to be mounted in a vicinity of the pyloric valve.

4. The apparatus according to claim 1, wherein the inflatable guide member is configured to be mounted within a duodenal bulb of the subject.

5. The apparatus according to claim 1, wherein the inflatable guide member is configured to be mounted outside of the small intestine.

6. The apparatus according to claim 1, wherein the inflatable guide member is configured to be mounted within the selected valve and to form the pressure seal, upon inflation, with the selected valve.

7. Apparatus for use with a biologically-compatible-fluid pressure source, comprising:
an inflatable guide member, configured to be mounted in a vicinity of a small intestinal site, and to form a pressure seal upon inflation;
an elongate carrier, configured to be slidably advanced through the guide member into a small intestine of the subject; and
an imaging capsule coupled to a distal portion of the carrier, the imaging capsule comprising:
an imaging element; and
a piston head configured to:
form a pressure seal with a wall of the small intestine, and
be advanced distally through the small intestine in response to pressure from the fluid pressure source.

8. The apparatus according to claim 1, wherein the capsule comprises a sample collection unit, configured to sample fluid or tissue of the small intestine.

9. The apparatus according to claim 1, wherein the apparatus comprises a sample collection unit coupled to the carrier and not an integral portion of the imaging capsule, wherein the collection unit is configured to sample fluid or tissue of the small intestine.

10. The apparatus according to claim 1, wherein the capsule is releasably coupled to the distal portion of the carrier, and wherein the capsule is configured to be released from the carrier while the capsule is in the small intestine.

11. The apparatus according to claim 1, wherein the piston head is at least 2 cm from the imaging element.

12. The apparatus according to claim 1, wherein the piston head is 3-5 cm from the imaging element.

13. The apparatus according to claim 1, wherein the piston head is positioned with respect to the imaging element such that distal motion of the imaging capsule causes cleaning of the imaging element by rubbing of the imaging element against the wall of the small intestine.

14. The apparatus according to claim 1, comprising a vent tube configured to facilitate passage of a fluid from (a) a site distal to the piston head to (b) a site proximal to the piston head.

15. The apparatus according to claim 1, wherein the inflatable guide member, is configured to be deflated following being mounted, and to subsequently be inflated at a site distal to where the inflatable guide member had been mounted, to an extent sufficient to form a pressure seal upon inflation.

16. A method for use with a biologically-compatible-fluid pressure source, comprising:
forming a pressure seal at a pressure seal site in a vicinity of a valve of a subject selected from the group consisting of: an ileocecal valve and a pyloric valve;
placing an elongate carrier distal to the pressure seal site, in a small intestine of the subject;
forming a pressure seal between a piston head coupled to the elongate carrier and a wall of the small intestine;
advancing the elongate carrier distally through the small intestine by applying pressure from the fluid pressure source to the piston head; and
imaging the small intestine from a distal portion of the carrier.

17. The method according to claim 16, wherein the selected valve includes the ileocecal valve, and wherein forming the pressure seal at the pressure seal site comprises forming the pressure seal in the vicinity of the ileocecal valve.

18. The method according to claim 16, wherein the selected valve includes the pyloric valve, and wherein forming the pressure seal at the pressure seal site comprises forming the pressure seal in the vicinity of the pyloric valve.

19. The method according to claim 16, wherein forming the pressure seal at the pressure seal site comprises forming the pressure seal within a duodenal bulb of the subject.

20. The method according to claim 16, wherein forming the pressure seal at the pressure seal site comprises forming the pressure seal outside of the small intestine.

21. The method according to claim 16, wherein forming the pressure seal site comprises forming the pressure seal within the selected valve.

22. A method for use with a biologically-compatible-fluid pressure source, comprising:
forming a pressure seal at a pressure seal site within a small intestine of a subject;
placing an elongate carrier distal to the pressure seal site, in the small intestine of the subject;
forming a pressure seal between a piston head coupled to the elongate carrier and a wall of the small intestine;
advancing the elongate carrier distally through the small intestine by applying pressure from the fluid pressure source to the piston head; and
imaging the small intestine from a distal portion of the carrier.

23. The method according to claim 16, comprising sampling fluid or tissue of the small intestine.

24. The method according to claim 16, wherein an imaging element for the imaging of the small intestine is coupled to the elongate carrier, and comprising releasing, in the small intestine, the imaging element from the elongate carrier.

25. The method according to claim 16, wherein imaging comprises imaging from a site at least 2 cm from the pressure seal between the piston head and the wall of the small intestine.

26. The method according to claim 16, wherein imaging comprises imaging from a site 3-5 cm from the pressure seal between the piston head and the wall of the small intestine.

27. The method according to claim 16, wherein an imaging element for the imaging of the small intestine is coupled to the elongate carrier, and comprising cleaning the imaging element by rubbing the imaging element against the wall of the small intestine.

28. The method according to claim 16, comprising facilitating passage of a fluid from (a) a site distal to the pressure seal between the piston head and the wall of the small intestine to (b) a site proximal to the pressure seal between the piston head and the wall of the small intestine.

29. The method according to claim 16, wherein the pressure seal site defines a first pressure seal site, and comprising removing the pressure seal at the first pressure seal site following forming the pressure seal at the first pressure seal site, and subsequently forming a pressure seal at a second pressure seal site distal to the first pressure seal site.
